(19) United States
(12) Patent Application Publication
(10) Pub. No.: US 2006/0200871 A1
(43) Pub. Date: Sep. 7, 2006

(54) Transgenic non-human animals with expanded mature b cell and plasma cell populations

(76) Inventors: Brian G. Van Ness, Lino Lakes, MN (US); Michael A. Linden, Falcon Heights, MN (US)

Correspondence Address: FISH & RICHARDSON P.C., PO BOX 1022, MINNEAPOLIS, MN, 55440-1022, US

(21) Appl. No.: 10/532,040

(22) PCT Filed: Oct. 20, 2003
(86) PCT No.: PCT/US03/33221

§ 371 Date: Dec. 30, 2005

Related U.S. Application Data
(60) Provisional application No. 60/419,800, filed on Oct. 21, 2002.

(51) Int. Cl. A01K 67/027; C12N 5/06

(52) U.S. Cl. 800/18; 435/354

(57) ABSTRACT

Transgenic non-human animals with expanded mature B cell and plasma cell populations are described, as well as methods of using the transgenic non-human animals to screen for pharmaceutically active agents and to produce targeted antibodies.

### TECHNICAL FIELD

The invention relates to transgenic non-human animals that exhibit expanded mature B cell and plasma cell populations. More particularly, the invention relates to transgenic non-human animals that express one or more anti-apoptotic polypeptides in the Bcl-2 family, under regulation by an immunoglobulin kappa light chain 3′ enhancer.

### BACKGROUND

Multiple myeloma is characterized by the clonal expansion of plasma cells in the bone marrow, and although there are effective therapeutic approaches, it remains an incurable disease. Difficulties in treating this disease include: 1) the resistance of the malignant plasma cells to induced cell death; 2) significant genetic heterogeneity resulting in a failure to define common genetic events; and 3) lack of good animal models. Induction of plasma cell tumors in mice has been achieved by injection of pristane (mineral oil); however, these tumors are solid plasmacytomas and not bone marrow malignancies. Human fetal bone implants have been transferred to SCID mice, where the implants support human myeloma cell growth, but the model is highly manipulated in a severely immunocompromised host.

### SUMMARY

The invention is based on the expression of an anti-apoptotic polypeptide in a transgenic non-human animal. Expression of the anti-apoptotic polypeptide is controlled by a tissue and developmentally regulated transcriptional enhancer, the immunoglobulin kappa light chain 3′ (K3′) enhancer, which restricts expression of the anti-apoptotic polypeptide to mature B cell/plasma cell populations. As a result, the transgenic non-human animals exhibit an expansion of mature B cell and plasma cell populations, but no increase in T cell or early B cell populations. The transgenic non-human animals also can express a proliferative oncogene such as ras or myc under control of a tissue and developmentally regulated transcriptional enhancer. Transgenic non-human animals that express both an anti-apoptotic polypeptide and the gene product of the proliferative oncogene typically exhibit plasma cell tumors.

In one aspect, the invention features a transgenic rodent (e.g., a mouse), the nucleated cells of which include a transgene, wherein the transgene includes an immunoglobulin kappa light chain 3′ enhancer sequence operably linked to a nucleic acid sequence that encodes an anti-apoptotic polypeptide in the Bcl-2 family. The transgenic rodent may exhibit an expanded plasma cell population and an expanded mature B cell population as compared with a corresponding wild-type rodent. The anti-apoptotic polypeptide can be selected from the group consisting of Bcl-2, Bcl-xL, Bcl-W, and Mcl-1. A human Bcl-xL polypeptide can be particularly useful. The transgene further can include a kappa promoter operably linked to a nucleic acid sequence encoding the anti-apoptotic polypeptide.

In another aspect, the invention features a transgenic rodent, the nucleated cells of which include (a) a first transgene that includes an immunoglobulin kappa light chain 3′ enhancer sequence operably linked to a nucleic acid sequence encoding an anti-apoptotic polypeptide in the Bcl-2 family; and (b) a second transgene that includes a B cell developmentally regulated transcriptional enhancer sequence operably linked to a proliferative oncogene nucleic acid sequence. The transgenic rodent can contain a plasma cell tumor. The proliferative oncogene nucleic acid sequence can be ras or myc. The B cell developmentally regulated transcriptional enhancer sequence can be an immunoglobulin kappa light chain 3′ enhancer sequence or an immunoglobulin heavy chain enhancer sequence. The anti-apoptotic polypeptide can be selected from the group consisting of Bcl-2, Bcl-xL, Bcl-W, and Mcl-1. A human Bcl-xL polypeptide can be particularly useful.

The invention also features progeny of such transgenic rodents, wherein the nucleated cells of the progeny include the transgene. In addition, the invention features isolated cells (e.g., plasma cells) of such transgenic rodents.

In yet another aspect, the invention features a method for identifying an agent that inhibits development of a plasma cell tumor. The method can include a) administering a test agent to a transgenic rodent described above, wherein the transgenic rodent develops a plasma cell tumor in the absence of pharmacological intervention; and b) determining if the test agent inhibits development of the plasma cell tumor in the transgenic rodent as compared with a corresponding transgenic rodent to which the test agent has not been administered.

The invention also features a method for identifying an agent for treating a plasma cell tumor. The method can include a) administering a test agent to a transgenic rodent described above, wherein the transgenic rodent exhibits a plasma cell tumor; and b) determining if the test agent slows tumor growth, stops tumor growth, reduces tumor size, or decreases plasma cell number in the transgenic rodent as compared with a corresponding transgenic rodent to which the test agent has not been administered.

In another aspect, the invention features a method for producing polyclonal antibodies. The method can include immunizing a transgenic rodent, the nucleated cells of which comprise a first transgene, the first transgene including an immunoglobulin kappa light chain 3′ enhancer sequence operably linked to a nucleic acid sequence encoding an anti-apoptotic polypeptide in the Bcl-2 family, wherein the transgenic rodent exhibits an expanded plasma cell population and mature B cell population as compared with a corresponding wild-type rodent; and harvesting the polyclonal antibodies. The transgenic rodent further can include a second transgene, the second transgene including a B cell developmentally regulated transcriptional enhancer sequence operably linked to a proliferative oncogene nucleic acid sequence, wherein the transgenic rodent exhibits a plasma cell tumor.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic depiction of the bcl-xL transgene. “3′KE” refers to 3′ kappa enhancer and “KP” refers to kappa promoter.

FIG. 2A is a graph showing isotype specific Ig levels in the serum of Bcl-XL mice and littermate controls (LMC). *, p<0.05; #, ng/ml. FIG. 2B is a pair of graphs showing TNP-Ficoll and TNP-CGG specific immunoglobulin levels in Bcl-XL and LMC mice. *, p<0.05.

FIG. 3A is a line graph showing survival curves of double transgenic, single transgenic, and LMC mice. FIG. 3B is a column graph showing survival of cultured splenocytes from double transgenic, single transgenic, and LMC mice.

### DETAILED DESCRIPTION

In general, the invention provides transgenic non-human animals that contain an anti-apoptotic transgene in their nucleated cells. Expression of the anti-apoptotic transgene can be regulated by, for example, the κ3′ enhancer, which is active late in B cell development. The transgenic non-human animals provided herein typically exhibit properties useful in the study of multiple myeloma, including an expansion of mature B cell and plasma cell populations, and an increase in immunoglobulin levels. Transgenic non-human animals with such properties can be used to determine whether or not a particular agent is useful for treating or preventing multiple myeloma. In addition, transgenic non-human animals with such properties can be used for producing targeted polyclonal antibodies.

Transgenic Non-Human Animals

As used herein, “transgenic non-human animal” includes founder transgenic non-human animals as well as progeny of the founders, progeny of the progeny, and so forth, provided that the progeny retain the transgene. Transgenic non-human animals can be, for example, farm animals such as pigs, goats, sheep, cows, horses, and rabbits, rodents such as rats, guinea pigs, and mice, and non-human primates such as baboons, monkeys, and chimpanzees. Transgenic rodents (e.g., transgenic mice) are particularly useful.

Tissues and cells (e.g., B cells or plasma cells) obtained from the transgenic non-human animals also are provided herein. The nucleated cells of the transgenic non-human animals provided herein contain a transgene that includes a nucleic acid sequence encoding an anti-apoptotic polypeptide in the Bcl-2 family. The nucleic acid sequence encoding the anti-apoptotic polypeptide can be a cDNA or can include introns or adjacent 5′- or 3′-untranslated regions (e.g., a genomic nucleic acid). As used herein, the term “anti-apoptotic polypeptide in the Bcl-2 family” refers to any chain of amino acids, regardless of post-translational modification, that has the ability to promote cell survival and that contains at least one of the four (e.g., one, two, three, or four) Bcl-2 homology. (BH) domains (BH1, BH2, BH3, and/or BH4). BH domains represent consensus sequences of anti-apoptotic polypeptides. See, PROSITE PDOC00829 from the Pfam Protein Families Database (Bateman et al. (2002) Nucleic Acids Res. 30:276-280). Amino acid substitutions, deletions, and insertions can be introduced into a known BH domain and the resulting polypeptide is an “anti-apoptotic polypeptide in the Bcl-2 family” provided that the polypeptide retains the ability to promote cell survival.

Suitable mammalian Bcl-2 family members include the Bcl-2, Bcl-xL, Bcl-W, Mcl-1, A1/BFL-1, BOO/DIVA, and NR13 polypeptides. See, e.g., Gross et al. (1999) Genes Dev. 13:1899-1911; and Cory and Adams (2002) Nature Reviews 2:647-656. Nucleic acid sequences encoding Bcl-2, Bcl-W, Mcl-1, or Bcl-xL polypeptides are particularly useful. Bcl-xL refers to the long isoform of Bcl-x, which typically is found in high levels in pre-B cells and in lower levels as the B cells mature. GenBank Accession Nos. L35049 and Z23115 L20121 provide the sequences of the mouse and human bcl-xL cDNAs, respectively.

The nucleic acid sequence encoding the anti-apoptotic polypeptide in the Bcl-2 family can be operably linked to a κ3′ enhancer sequence. See, Fulton and Van Ness (1993) Nucleic Acids Res. 21:4941-4947 for a description of κ3′ enhancer elements. The nucleic acid sequence of the κ3′ enhancer sequence can be found, for example, in GenBank under Accession No. X15878 (Meyer and Neuberger (1989) EMBO J. 8:1959-1964). As used herein, “operably linked” refers to positioning of a regulatory element relative to a nucleic acid sequence encoding a polypeptide in such a way as to permit or facilitate expression of the encoded polypeptide. In the transgenes disclosed herein, for example, the κ3′ enhancer can be positioned 3′ or 5′ relative to the nucleic acid encoding the anti-apoptotic polypeptide, and can be positioned within the transgene in either the 5′ to 3′ or the 3′ to 5′ orientation. Typically, the 5′ end of the κ 3′ enhancer is positioned less than 5 kb (e.g., 0 to 1 kb, 1 to 2 kb, 2 to 3 kb, 3 to 4 kb, or 4 to 5 kb) from the nucleic acid sequence encoding the anti-apoptotic polypeptide.

Transgenes of the invention can include additional regulatory elements, including, without limitation, promoters, inducible elements, or other upstream promoter elements, operably linked to the nucleic acid sequence encoding the polypeptide. In some embodiments, a tissue specific promoter is operably linked to the nucleic acid sequence encoding the anti-apoptotic polypeptide. Suitable tissue specific promoters can result in preferential expression of a nucleic acid transcript in cells of the B cell lineage and include, for example, the kappa V-region promoter and the kappa germline promoter. See, Fulton and Van Ness (supra) for a description of the kappa promoters. In other embodiments, a promoter that facilitates the expression of a nucleic acid molecule without significant tissue- or temporal-specificity can be used. For example, the β-globin promoter can be used, as well as viral promoters such as the herpes virus thymidine kinase (TK) promoter, the SV40 promoter, or a cytomegalovirus (CMV) promoter.

In some embodiments, the transgene can include a tag sequence that encodes a “tag” designed to facilitate subsequent manipulation of the encoded polypeptide (e.g., to facilitate localization or detection). Tag sequences can be inserted in the nucleic acid sequence encoding the anti-apoptotic polypeptide such that the encoded tag is located at either the carboxyl or amino terminus of the anti-apoptotic polypeptide. Non-limiting examples of encoded tags include green fluorescent protein (GFP), glutathione S-transferase (GST), and Flag tag (Kodak, New Haven, Conn.)

Various techniques known in the art can be used to introduce transgenes into non-human animals to produce founder lines, in which the transgene is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82:6148-1652), gene targeting into embryonic stem cells (Thompson et al. (1989) Cell 56:313-321), electroporation of embryos (Lo (1983) Mol. Cell. Biol. 3:1803-1814), and in vitro transformation of somatic cells, such as cumulus or mammary cells, followed by nuclear transplantation (Wilmut et al. (1997) Nature 385:810-813; and Wakayama et al. (1998) Nature 394:369-374). For example, fetal fibroblasts can be genetically modified to express an anti-apoptotic polypeptide, and then fused with enucleated oocytes. After activation of the oocytes, the eggs are cultured to the blastocyst stage. See, for example, Cibelli et al. (1998) Science 280:1256-1258. Standard breeding techniques can be used to create animals that are homozygous for the anti-apoptotic transgene from the initial heterozygous founder animals. Homozygosity is not required, however, as the phenotype can be observed in hemizygotic animals (see the Examples herein).

Once transgenic non-human animals have been generated, expression of an anti-apoptotic polypeptide can be assessed using standard techniques. Initial screening can be accomplished by Southern blot analysis to determine whether or not integration of the transgene has taken place. For a description of Southern analysis, see sections 9.37-9.52 of Sambrook et al., 1989, Molecular Cloning, A Laboratory Manual, second edition, Cold Spring Harbor Press, Plainview; N.Y. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis (1992) Genetic Engineering News 12:1; Guatelli et al. (1990) Proc. Natl. Acad. Sci. USA 87:1874-1878; and Weiss (1991) Science 254:1292-1293.

Expression of a nucleic acid sequence encoding an anti-apoptotic polypeptide (e.g., a Bcl-xL polypeptide) in the tissues of transgenic non-human animals can be assessed using techniques that include, without limitation, Northern blot analysis of tissue samples obtained from the animal (e.g., bone marrow or spleen tissue), in situ hybridization analysis, Western analysis, immunoassays such as enzyme-linked immunosorbent assays, and reverse-transcriptase PCR (RT-PCR). As described herein, expression of Bcl-xL can result in tissue restricted expression of the transgene in mature B cell/plasma cell populations, and one or more of the following characteristics: an expansion of B cells in the marrow, an increase in post switched B cells (multiple isotypes, including IgG, IgA, and IgE), an increase in the plasma cell content in the spleen, an increase in IgG⁺ cells in the marrow, blood lymphocytosis, increased IgH and IgL serum content, and proteinuria. The kidney may have hyaline tubular casts, foci of abnormal plasma cells, and glomerular amyloid deposition secondary to Ig accumulation. In some embodiments, the transgenic animals can exhibit bone fractures, an event that is secondary in plasma cell neoplasia It is understood that a particular phenotype in a transgenic animal typically is assessed by comparing the phenotype in the transgenic animal to the corresponding phenotype exhibited by a control non-human animal that lacks the transgene.

In one embodiment, the transgenic non-human animals further includes a second transgene that contains a nucleic acid sequence of a proliferative oncogene such as myc or ras. The nucleic acid sequence of human myc, for example, has GenBank Accession No. X00364. The nucleic acid sequence of human Nras has GenBank Accession No. NM_{—}002524. The nucleic acid sequence of the proliferative oncogene also can be operably linked to a tissue and/or developmentally regulated transcriptional enhancer. For example, the nucleic acid sequence of human myc or ras can be operably linked to the κ 3′ enhancer or to the μ heavy chain intron enhancer. The μ heavy chain enhancer is active early in B cell development and throughout all remaining developmental stages of the B cell (Gillies et al. (1983) Cell 33:717-728; Banerji et al. (1983) Cell 33:729-740; and Grosschedl and Baltimore (1985) Cell 41:885-897). The second transgene also can include other regulatory elements as discussed above (e.g., a tissue-specific promoter).

Expression of a proliferative oncogene gene product and an anti-apoptotic polypeptide can enhance tumor pathology in a transgenic non-human animal as compared with a control transgenic non-human animal expressing only the anti-apoptotic polypeptide. “Control” typically refers to the same background strain and same species of transgenic non-human animal, e.g., both transgenic non-human animals are rats or both are mice. As described herein, transgenic mice expressing a myc or ras gene product and an anti-apoptotic polypeptide (e.g., a Bcl-xL polypeptide) can exhibit clonal expansion and tumor formation (e.g., plasma cell tumors such as bone marrow malignancies or solid plasmacytomas). Plasma cells or mature B cells removed from such transgenic mice can have an increased longevity in culture (e.g., the cells may be immortalized). Such cells can be used to, for example, screen for pharmaceutically active agents.

A transgenic non-human animal expressing both an anti-apoptotic polypeptide in the Bcl-2 family (e.g., a Bcl-xL polypeptide) and a proliferative oncogene can be produced by, for example, crossing (a) a transgenic non-human animal overexpressing an anti-apoptotic polypeptide with (b) a transgenic non-human animal overexpressing the product of the proliferative oncogene. Transgenic mice overexpressing myc are described in, for example, Schmidt et al. (1988) Proc. Natl. Acad. Sci. USA 85:6047-6051, and Adams et al. (1985) Nature 318:533-538, while transgenic mice overexpressing ras are described in, for example, Cardiff et al. (1993) Am. J. Path. 142:1199-1207. Alternatively, a single line of transgenic non-human animals can be produced by initially preparing the non-human animals using the appropriate transgenes.

Antibody Production

Transgenic animals described herein can be used to produce polyclonal antibodies against a protein of interest. In general, a polypeptide of interest can be produced recombinantly, by chemical synthesis, or by purification of the native protein, and then used to immunize a transgenic non-human animal of the invention. Adjuvants can be used to increase the immunological response, depending on the species of transgenic non-human animal. Suitable adjuvants can include, for example, Freund's adjuvant (complete and incomplete), mineral gels such as aluminum hydroxide, surface-active substances such as lysolecithin, pluronic polyols, polyanions, peptides, oil emulsions, keyhole limpet hemocyanin (KLH), and dinitrophenol. Polyclonal antibodies are heterogeneous populations of antibody molecules that are specific for a particular antigen (e.g., a polypeptide of interest), and can be harvested from the sera of immunized animals (e.g., immunized transgenic animals containing an anti-apoptotic transgene in their nucleated cells).

Once produced, antibodies or fragments thereof can be tested for binding affinity to the protein of interest by standard immunoassay methods including, for example, enzyme linked immunosorbent assay (ELISA) techniques or radioimmunoassays. See, e.g., Short Protocols in Molecular Biology, Chapter 11, Green Publishing Associates and John Wiley & Sons, ed. Ausubel et al., 1992.

Screening for Pharmaceutically Active Agents

The transgenic non-human animals described herein can be used to screen for (a) pharmaceutically active agents that inhibit the development of plasma cell tumors, or (b) agents that can be used for treating plasma cell tumors (e.g., reducing plasma cell number, slowing or stopping tumor growth, or reducing tumor size). For example, in embodiments in which agents are screened for treatment of plasma cell tumors, a candidate agent can be administered to a transgenic non-human animal that has developed the tumors, and plasma cell number, tumor size, or other characteristics can be monitored in the transgenic non-human animal and compared with the same characteristics of a corresponding transgenic non-human animal to which the test agent has not been administered.

Suitable candidate agents can include, for example, chemical compounds, mixtures of chemical compounds, biological macromolecules (e.g., polypeptides), or biological materials such as extracts of bacteria, plants, fungi, and animals. A variety of techniques can be used for random and directed synthesis of a wide variety of organic compounds and biomolecules, including synthesis of randomized oligonucleotides and oligopeptides. Natural or synthetically produced libraries and compounds can be modified using chemical or physical techniques known in the art, and such techniques can be used to produce combinatorial libraries. Known pharmacological agents may be subjected to directed or random chemical modifications, (e.g., acylation, alkylation, esterification, or amidification) to produce structural analogs.

Agents can be formulated into pharmaceutical compositions by admixture with pharmaceutically acceptable non-toxic excipients or carriers and administered to the transgenic non-human animals by any route of administration. For example, parenteral routes such as subcutaneous, intramuscular, intravascular, intradermal, intranasal, inhalation, intrathecal, or intraperitoneal administration, and enteral routes such as sublingual, oral, or rectal administration can be used;

A number of methods can be used to determine whether or not an agent alters a particular phenotype exhibited by a transgenic non-human animal, including, without limitation, biochemical, histological, or behavioral assays. Suitable biochemical assays (e.g., fluorescence activated cell sorting (FACS) analysis, ELISA assays) are known in the art. Histological assays also can be used to determine whether on not an agent affects a particular phenotype exhibited by a transgenic non-human animal (e.g., hyaline tubular casts or foci of abnormal plasma cells in the kidney).

It is understood that when comparing phenotypes to assess the effects of a test agent, a statistically significant difference indicates that that particular test agent, test dosage, or test duration warrants further study. Typically, a difference in phenotypes is considered statistically significant at p<0.05 with an appropriate parametric or non-parametric statistic, e.g., Chi-square test, Student's t-test, Mann-Whitney test, or F-test.

The invention will be further described in the following examples, which do not limit the scope of the invention described in the claims.

### EXAMPLES

### Example 1

### Production and Characterization of Bcl-XL Transgenic Mice

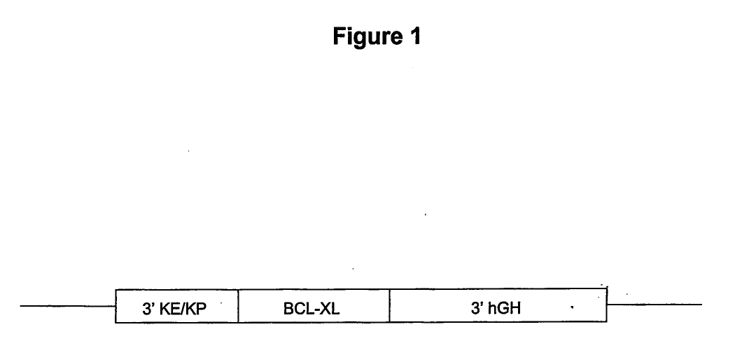

Transgenic mice were generated to overexpress BC-X_{L} in their B-lymphoid cells. To prepare the transgene vector, the 3′ kappa enhancer and V_{κ}21E promoter (3′KE/KP) were isolated from K3′E.KP.LUC (Fulton and Van Ness (1994) Nucl. Acids Res. 22:4216-4123). The Eμ and TK promoter were excised from a Bcl-XL construct containing murine Bcl-XL cDNA linked to sequences encoding the FLAGS epitope (Fang et al. (1996) Immunity 4:291-299) and were replaced with the 3′KE/KP sequence. In addition, a human growth hormone (hGH) minigene sequence (Anderson et al. (1993) EMBO J. 12:1671-1680) was placed at the 3′ end of the construct to provide RNA splicing and processing. The resulting p3′KE/13cl-XL transgene construct (FIG. 1) was purified by CsCl centrifugation, linearized with Not I and Ase I, and injected into male pronuclei of FVB/N mouse embryos.

Mice were maintained in standard pathogen free facilities and analyzed between the ages of 4-10 weeks. Southern blotting was used to identify founders and estimate transgene copy number. Genomic DNA was isolated from tailsnips was obtained as previously described (O'Brien et al. (1997) Mol. Cell. Biol. 17:3477-3487). Proteinase K (20 mg/mL) was added to the lysis solution, and the samples were incubated overnight at 55° C. to ensure complete sample digestion. Ten jig of genomic DNA was digested with Bgl II (Invitrogen Life Technologies, Carlsbad, Calif.), and a Bcl-XL cDNA was used as a probe for Southern blotting. To determine clonality of isolated samples, 10 μg of genomic DNA was digested with Bam HI (Invitrogen Life Technologies) and probed with DNA corresponding to the kappa locus.

For genotyping, 100 ng of genomic DNA was amplified by PCR using the following primer sets: Bcl-XL—FLAG, 5′-GACTACAAGGACGACGATGACAAG-3′ (SEQ ID NO: 1), and RBCLDOWN, 5′-AGTGGATGGTCAGTGTCTGGTCAC-3′ (SEQ ID NO:2); c-Myc—1MYC, 5′-CAGCTGGCGTAATAGCGAAGAG-3′ (SEQ ID NO:3), and 2MYC 5′-CTGTGACTGGTGAGTACTCAACC-3′ (SEQ ID NO:4); IL-2—1IL-2, 5′-CTAGGCCACAGAATTGAAAGATCT-3′ (SEQ ID NO:5), and 2IL-2 5′-GTAGGTGGAAATTCTAGCATCATCC-3′ (SEQ ID NO:6).

These analyses revealed that five independent transgenic founders were produced. Of the five founders, four demonstrated germline transmission of the transgene, and three founder lines were maintained and characterized. All three of the characterized founder lines demonstrated similar phenotypes. All experiments were performed with heterozygous animals.

To characterize transgene expression, RNA and protein were isolated from the marrow, spleen, liver, kidney, lung, heart, lymph, and thyroid of a six-week-old BCl-XL mouse. RT-PCR was used to detect expression of the transgene in the various tissues. RNA samples were isolated using TRIzol® and were treated with DNase I (Invitrogen Life Technologies). cDNA was synthesized using Superscript™ RT (Invitrogen, Rockville, Md.), and template cDNA was amplified using the BC1-XL primers used for genotyping. Actin primers were used as a control (ACTINUP, 5′-CCTAAGGCCAACCGTGAAAAG-3′, SEQ ID NO:7; and ACTINDOWN 5′-TCTTCATGGTGCTAGGAGCCA-3′, SEQ ID NO:8). In addition, western blotting was performed as previously described (Liu et al. J. Immunol. 165:7058-7063 (2000)) using the following antibodies: anti-Bcl-XL (Santa Cruz Biotech, Santa Cruz, Calif.), anti-FLAG (Sigma, St. Louis, Mo.), anti-actin (Santa Cruz Biotech), and anti-mouse Ig (Amersham, Cleveland, Ohio).

Since the transgenic Bcl-XL was tagged with the FLAG® epitope, both endogenous and transgenic Bcl-XL levels were determined. Transgenic Bcl-XL levels were highest in the marrow, spleen, and lymph nodes. Low levels of transcripts also were detected in other tissues, likely due to transgene-expressing lymphocytes in peripheral circulation that migrated to those locations, including microscopic foci of lymphocytes that were detected upon histopathological examination. Western blotting revealed that transgenic Bcl-XL expression at the protein level was confined to the marrow, spleen, and lymph. Two isoforms of Bcl-XL were observed—Bcl-XL, and the alternatively spliced Bcl-XΔTM isoform (loss of transmembrane domain; Fang et al. (1994) J. Immunol. 153:4388-4398).

FACS was used in conjunction with RT-PCR to determine which populations of lymphocytes exhibited 3′KE activity. The following monoclonal antibodies were used for cell staining: anti-CD-3, anti-B220, anti-CD138, anti-IgM, anti-IgD, and anti-HSA (all antibodies from BD Pharmingen, San Diego, Calif., except anti-HSA, which was obtained from T. Waldschmidt, University of Iowa). Standard flow cytometric analysis was carried out on a FACSCalibur machine (BD Pharmingen), and data were collected and analyzed using Cellquest Pro (BD Pharmingen). Stained cells were sorted on a FACSVantage machine (BD Pharmingen).

Marrow cells from Bcl-XL mice were stained with anti-kappa, anti-B220, anti-IgM-FITC (surface) and/or anti-IgM-PE (cytoplasmic) antibodies. Spleen cells from Bcl-XL mice were stained with anti-B220, anti-CD3, anti-IgM, and/or anti-IgD antibodies. Transgene transcripts were detected in the B220⁺/kappa⁻, B220⁺/kappa⁺, and cIgM^{hi}/sIgM^{lo} marrow populations. Transgenic transcripts were detected in the B220⁺/CD3⁻, IgM^{hi}IgD^{hi}B220⁺ and IgM^{lo}Ig^{hi}B220⁺ spleen populations but not detected in the B220⁻/CD3⁻ or IgM^{hi}IgD^{lo}B₂₂₀⁺ spleen populations. This analysis demonstrated that 3′KE activity and transgene expression were detectable in bone marrow plasma cells and in B-cells, including immature/transitional B-cells of the spleen, but that transgene expression was not detectable in T-cells.

To determine whether the Bcl-XL transgenic mice had altered lymphocyte compartments, samples of peripheral blood from age-matched Bcl-XL mice (n=3) and LMC animals (n=2) were analyzed on a Hemavet® automated cell counter. All Bcl-XL animals demonstrated lymphocytosis compared to LMC, with an average count of 13.2±2.26 K/μL versus 3.5±0.21 K/μL (P=0.02).

To further characterize the differences in the lymphocyte populations, samples of cells from LMC or Bcl-XL mice were stained with fluorescently-labeled antibodies corresponding to different B- and T-lymphocyte cell surface markers, and analyzed by flow cytometry. Splenocytes from age-matched LMC and Bcl-XL mice were stained with anti-B220 and anti-CD3 antibodies. The percentages of B- and T-lymphocytes were similar in both groups. Since the 3′KE is not strongly active until the immature and mature stages of B-cell development, altered lymphocyte populations were not expected until those stages and beyond. Indeed, no differences in the BP-1⁺ pre-B cell populations were observed between Bcl-XL and LMC mice. Marrow cells from Bcl-XL transgenic and LMC mice were stained with anti-B220, anti-IgM, and anti-HSA antibodies, and spleen cells from Bcl-XL transgenic and LMC mice were stained with anti-B220, anti-IgD, and anti-IgM antibodies. The Bcl-XL transgenic animals showed a significant increase in the B220⁺/IgM^{hi}/IgD^{hi} immature/transitional B-cells of the spleen, as well as the B220⁺/IgM⁺ immature (HSA^{high}) and mature (HSA^{low}) B-cells of the marrow. Marrow cells also were stained with anti-B220 and anti-CD138 antibodies to examine the plasma cell (PC) compartments of LMC and Bcl-XL transgenic mice. No significant differences were observed between the CD138^{hi}B220^{lo}PC populations of the two groups.

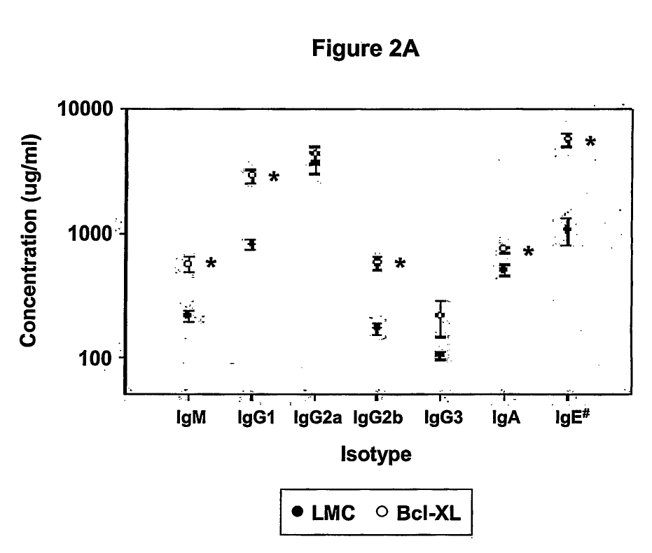

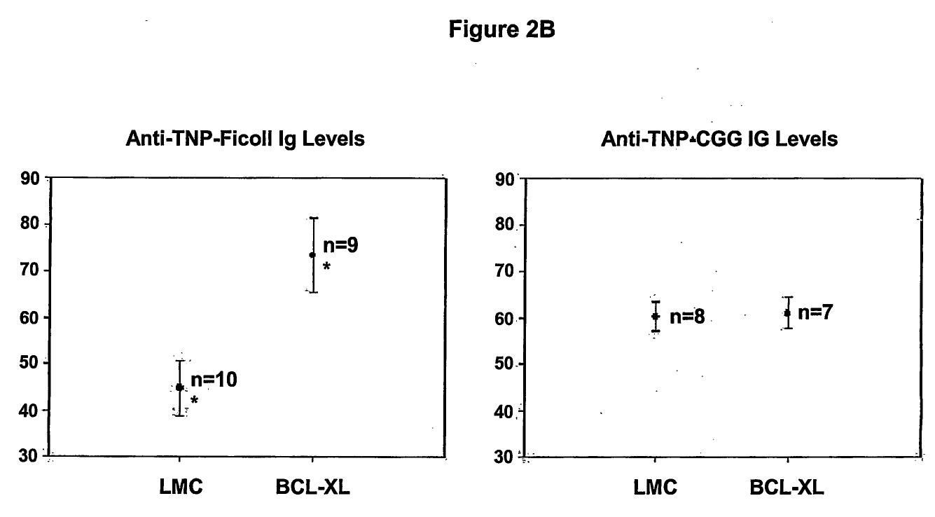

To evaluate serum immunoglobulin levels, serum samples were collected from Bcl-XL and LMC mice for western blotting with an anti-mouse Ig antibody. These studies demonstrated that total amounts of heavy and light chain Ig proteins were significantly elevated in the Bcl-XL mice as compared to LMC, to the extent that Ig proteins were excreted in the urine of Bcl-XL mice. ELISAs were conducted as previously described (Burns et al. (2003) Bone Marrow Transplant. 32:177-186) to determine isotype-specific serum Ig levels. Bcl-XL animals demonstrated significantly (P<0.05) elevated levels of the IgM, IgG1, IgG2b, IgA, and IgE isotypes as compared to LMC (FIG. 2A). Serum samples were analyzed by serum protein electrophoresis and two-dimensional gel electrophoresis, and although the gamma globulin fractions in the Bcl-XL mice were expanded, no clonal spikes were observed in any of the samples.

Given the putative apoptosis-resistant B-lymphocytes and elevated serum Ig levels in the Bcl-XL animals, Bcl-XL animals were examined to evaluate their response to antigenic challenge and to determine whether they make more antigen-specific Ig than LMC. BCl-XL and LMC animals were immunized with either TNP-Ficoll, a T-cell independent antigen, or TNP-CGG, a T-cell dependent antigen. Primary immunizations were given in complete Freund's adjuvant (CFA), and secondary immunizations were given fourteen days later in incomplete Freund's adjuvant (IFA). Serum samples were collected fourteen days after the secondary immunization, a time point when maximum antibody production was expected. ELISAs were performed to estimate antigen-specific Ig levels. These experiments revealed that the Bcl-XL mice produced 65% more TNP-Ficoll specific Ig than LMC (P<0.05), although Bcl-XL and LMC animals produced similar amounts of TNP-CGG specific Igs (FIG. 2B). Thus, Bcl-XL mice produced ore T-cell independent antigen-specific Ig than LMC.

For histological analyses, mice were euthanized, necropsied, and fixed in 10% formalin (Sigma, St. Louis, Mo.). Selected tissues (kidneys, spleen, lymph nodes, liver, lung, heart, gastrointestinal tissue, and long and flat bones) were embedded in paraffin and sectioned at 3-5 μm. Tissue sections were either stained with hematoxylin and eosin or prepared for immunohistochemical analysis (IHC). For IHC, tissue sections were steamed for 30 minutes in 1 mM EDTA, pH 8.0, and blocked with 3% hydrogen peroxide, Avidin/Biotin Block (Vector, Burlingame, Calif.), and Dako Protein Block (Dako, Carpenteria, Calif.). The sections were incubated overnight with anti-B220 or anti-CD138 antibodies (BD Pharmingen) at 4° C., and then incubated with Strepavidin HRP enzyme conjugate (Dako, Carpenteria, Calif.) for 15 minutes. Vector Nova Red Substrate (Vector, Burlingame, Calif.) was used for color determination. Sections were counterstained with Mayer's hematoxylin.

Hematoxylin and eosin staining revealed no abnormalities in the LMC mice studied. In contrast, histologic analysis of tissue sections from Bcl-XL mice revealed profound lymphocytic pathology in the three founder lines studied, and the incidence of microscopic lesions correlated directly with transgene copy number and age of the animal. These histopathological changes were detected in all Bcl-XL mice studied, ranging between 3 and 24 months of age.

The Bcl-XL mice demonstrated pathological changes in the kidney, and mice that had urine Ig levels detectable by Western blot analysis demonstrated the most pronounced pathology. Furthermore, a number of animals developed perivascular foci of lymphocytes in the kidney. Immunostaining of kidney sections with anti-CD138 or anti-B220 antibodies showed that the foci stained intensely for surface CD138.but did not stain for surface B220, consistent with the cell surface phenotype of PCs. The presence of PC foci in the kidney and in other tissues indicated that the total amount of Ig protein filtered by the kidney was increased in the transgenic animals. As a result, hyaline renal tubular casts (Ig protein deposits in renal tubules) were detected in a number of animals. In addition, foci of abnormal plasma cells (including Mott cells) with atypical nuclei were observed in renal tubules of transgenic animals; the cells found in these foci were consistent with PC neoplasia. Two of the transgenic animals with extensive renal pathology also exhibited glomerular amyloid deposition, as determined by Congo red stain.

Histopathological changes also were noted in the marrow and other lymphoid organs of the Bcl-XL mice. Evaluation of bone marrow sections revealed that while marrow architecture was intact, the percentage of lymphocytes comprising the marrow cavity was significantly increased as compared to LMC. Additionally, nests of PCs were found in the marrow of mice greater than one year of age.

Radiographs of multiple mice showed a lack of diffuse demineralization or focal lesions in the Bcl-XL or LMC mice. Some of the mice demonstrated sheets of PCs in their lymph nodes. Foci of CD 138⁺ PCs and B220⁺ lymphocytes also were detected in other non-lymphoid soft tissues, including liver and lung in BCl-XL animals. Radiographs of mice with confirmed PC pathology also revealed distinct fractures in the femora (proximal to the femoral head) of two mice and the scapula of one mouse.

Lymph node involvement was unappreciable, except for a single mouse with popliteal lymph node enlargement (the node contained sheets of plasma cells). In addition, three transgenic mice from two separate founder lines exhibited significant gall bladder distention upon necropsy. The fluid contained within the gall bladders of these animals contained significant amounts of heavy and light chain Ig proteins. Multiple other animals demonstrated foci of PCs in multiple soft tissues, including lung and liver.

### Example 2

### Production and Characterization of Bcl-XL/c-Myc Double Transgenic Mice

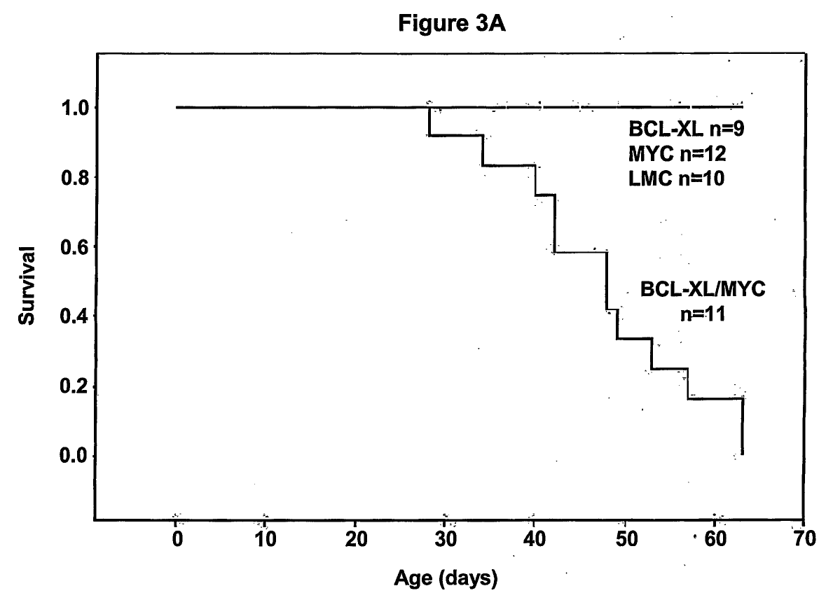

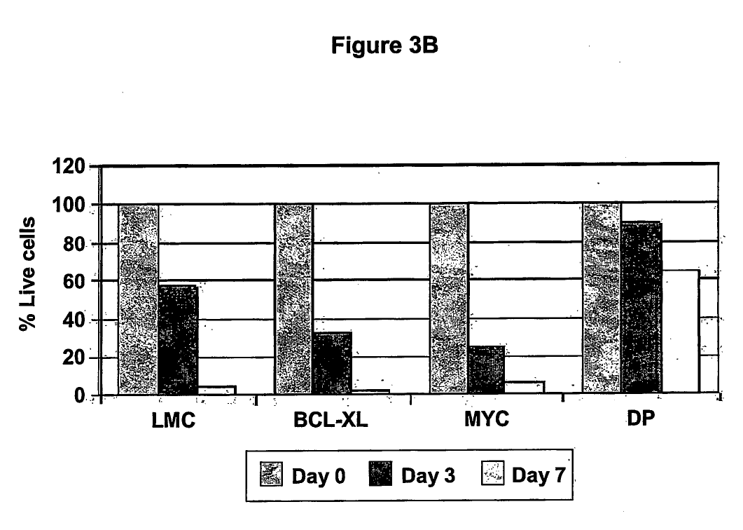

The 3′KE/Bcl-XL mouse was crossed with the Eμ/c-Myc mouse to evaluate the cooperativity of Bcl-XL and c-Myc under control of the 3′KE and Eμ promoters, respectively. Coexpression of anti-apoptotic Bcl-XL and oncogenic c-Myc in the double transgenic animals was highly fatal, and 50 percent of the double transgenic mice died of a lymphoproliferative disorder at 6.7 weeks (FIG. 3A). Mononuclear spleen cells from all four genotypes comprising the F1 progeny were cultured in RPMI 1640 medium supplemented with 10% FCS, 2 mM L-glutamine, 100 U/mL penicillin, and 100 μg/mL streptomycin (all from Invitrogen Life Technologies). Viable cells were counted at days 3 and 7. The majority of cells from single transgenic mice and LMC were dead at day 7, while more than 60% of the double transgenic cells were viable (FIG. 3B). Furthermore, more than 60% of splenocytes from the double transgenic mice were still viable after four weeks in culture.

Samples of peripheral blood from age-matched double transgenic mice (n=2) and LMC mice (n=2) were analyzed on a Hemavet® automated cell counter. The transgenic animals demonstrated lymphocytosis as compared to the LMC animals, with an average count of 79.16±3.08 K/μL versus 6.32±2.74 K/μL (P=0.02). Staining with anti-kappa and anti-B220 antibodies showed that 52.78±6.23 % of the peripheral blood mononuclear cells from the double transgenic mice were B220⁺/kappa⁺, versus 5.38±0.24 % of the peripheral blood mononuclear cells from LMC. In addition, serum Ig levels were detected by Western blotting. While the serum of double transgenic mice contained more heavy and light chain Ig proteins than the serum of LMC or c-Myc mice, the BCl-XL mice had the most elevated levels of serum Ig proteins.

Necropsies revealed that all double transgenic animals demonstrated a very pronounced splenomegaly. In addition, multiple light-colored nodules up to 2 mm in diameter were observed across the surface of the livers of the double transgenic animals. Histological sections of kidneys, spleen, lymph nodes, liver, lung, heart, gastrointestinal tissue, thymus, pancreas, and long and flat bones were stained with hematoxylin and eosin. These studies revealed an infiltration of mononuclear cells with large, round to polygonal euchromatic nuclei and a low mitotic rate in the kidneys, spleen, lymph node, liver, lung, heart, thymus, and pancreas. Similar mononuclear cells were seen in the sternum. Radiographs of the mice showed osteolytic lesions in the long bones of the double transgenic animals. When these areas of bone were sectioned and stained, they were found to contain solid PC tumors. The trabecules within the bones were lysed, revealing that the marrow cavities were filled with solid high-grade tumors. In some of the bones, the tumor cells penetrated the corticalis and entered the surrounding soft tissue. Southern blot analysis was used to examine the clonality of samples isolated from two double transgenic mice. Clonally-related populations were detected in samples of blood, marrow, spleen, and liver nodule.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A transgenic rodent, the nucleated cells of which comprise a transgene, said transgene comprising an immunoglobulin kappa light chain 3′ enhancer sequence operably linked to a nucleic acid sequence encoding an anti-apoptotic polypeptide in the Bcl-2 family, wherein said transgenic rodent exhibits expanded plasma cell and mature B cell populations as compared with a corresponding wild-type rodent.

2. The transgenic rodent of claim 1, wherein said transgenic rodent is a mouse.

3. The transgenic rodent of claim 1, wherein said anti-apoptotic polypeptide is selected from the group consisting of Bcl-2, Bcl-xL, Bcl-W, and Mcl-1.

4. The transgenic rodent of claim 1, wherein said anti-apoptotic polypeptide is a human Bcl-xL polypeptide.

5. Progeny of the transgenic rodent of claim 1, wherein the nucleated cells of said progeny comprise said transgene.

6. An isolated cell of the transgenic rodent of claim 1.

7. The cell of claim 6, wherein said cell is a plasma cell.

8. The transgenic rodent of claim 1, wherein said transgene further comprises a kappa promoter operably linked to a nucleic acid sequence encoding said anti-apoptotic polypeptide.

9. A transgenic rodent, the nucleated cells of which comprise:
(a) a first transgene comprising an immunoglobulin kappa light chain 3′ enhancer sequence operably linked to a nucleic acid sequence encoding an anti-apoptotic polypeptide in the Bcl-2 family; and
(b) a second transgene comprising a B cell developmentally regulated transcriptional enhancer sequence operably linked to a proliferative oncogene nucleic acid sequence, wherein said transgenic rodent contains a plasma cell tumor.

10. The transgenic rodent of claim 9, wherein said proliferative oncogene nucleic acid sequence is ras.

11. The transgenic rodent of claim 9, wherein said proliferative oncogene nucleic acid sequence is myc.

12. The transgenic rodent of claim 9, wherein said B cell developmentally regulated transcriptional enhancer sequence is an immunoglobulin kappa light chain 3′ enhancer sequence.

13. The transgenic rodent of claim 9, wherein said B cell developmentally regulated transcriptional enhancer sequence is an immunoglobulin heavy chain enhancer sequence.

14. The transgenic rodent of claim 9, wherein said anti-apoptotic polypeptide is selected from the group consisting of Bcl-2, Bcl-xL, Bcl-W, and Mcl-1.

15. The transgenic rodent of claim 9, wherein said anti-apoptotic polypeptide is a human Bcl-xL polypeptide.

16. Progeny of the transgenic rodent of claim 9, wherein said progeny comprise said first transgene and said second transgene.

17. An isolated cell of the transgenic rodent of claim 9.

18. The cell of claim 17, wherein said cell is a plasma cell.

19. A method for identifying an agent that inhibits development of a plasma cell tumor, said method comprising:
a) administering a test agent to a transgenic rodent, the nucleated cells of which comprise
(i) a first transgene comprising an immunoglobulin kappa light chain 3′ enhancer sequence operably linked to a nucleic acid sequence encoding an anti-apoptotic polypeptide in the Bcl-2 family; and
(ii) a second transgene comprising a B cell developmentally regulated transcriptional enhancer sequence operably linked to a proliferative oncogene nucleic acid sequence, wherein said transgenic rodent develops a plasma cell tumor in the absence of pharmacological intervention; and
b) determining if said test agent inhibits development of said plasma cell tumor in said transgenic rodent as compared with a corresponding transgenic rodent to which said test agent has not been administered.

20. A method for identifying an agent for treating a plasma cell tumor, said method comprising:
a) administering a test agent to a transgenic rodent, the nucleated cells of which comprise
(i) a first transgene comprising an immunoglobulin kappa light chain 3′ enhancer sequence operably linked to a nucleic acid sequence encoding an anti-apoptotic polypeptide in the Bcl-2 family; and
(ii) a second transgene comprising a B cell developmentally regulated transcriptional enhancer sequence operably linked to a proliferative oncogene nucleic acid sequence, wherein said transgenic rodent exhibits a plasma cell tumor; and
b) determining if said test agent slows tumor growth, stops tumor growth, reduces tumor size, or decreases plasma cell number in said transgenic rodent as compared with a corresponding transgenic rodent to which said test agent has not been administered.

21. A method for producing polyclonal antibodies, said method comprising immunizing a transgenic rodent, the nucleated cells of which comprise a first transgene, said first transgene comprising an immunoglobulin kappa light chain 3′ enhancer sequence operably linked to a nucleic acid sequence encoding an anti-apoptotic polypeptide in the Bcl-2 family, wherein said transgenic rodent exhibits an expanded plasma cell and mature B cell population as compared with a corresponding wild-type rodent; and harvesting said polyclonal antibodies.

22. The method of claim 21, wherein said transgenic rodent further comprises a second transgene, said second transgene comprising a B cell developmentally regulated transcriptional enhancer sequence operably linked to a proliferative oncogene nucleic acid sequence, wherein said transgenic rodent exhibits a plasma cell tumor.


SEQUENCE LISTING (8 sequences)

SEQ ID NO: 1 - DNA, 24 bp, Artificial Sequence
  Primer
gactacaaggacgacgatgacaag

SEQ ID NO: 2 - DNA, 24 bp, Artificial Sequence
  Primer
agtggatggtcagtgtctggtcac

SEQ ID NO: 3 - DNA, 22 bp, Artificial Sequence
  Primer
cagctggcgtaatagcgaagag

SEQ ID NO: 4 - DNA, 23 bp, Artificial Sequence
  Primer
ctgtgactggtgagtactcaacc

SEQ ID NO: 5 - DNA, 24 bp, Artificial Sequence
  Primer
ctaggccacagaattgaaagatct

SEQ ID NO: 6 - DNA, 25 bp, Artificial Sequence
  Primer
gtaggtggaaattctagcatcatcc

SEQ ID NO: 7 - DNA, 21 bp, Artificial Sequence
  Primer
cctaaggccaaccgtgaaaag

SEQ ID NO: 8 - DNA, 21 bp, Artificial Sequence
  Primer
tcttcatggtgctaggagcca